(19) United States
(12) Patent Application Publication
(10) Pub. No.: US 2006/0024239 A1
(43) Pub. Date: Feb. 2, 2006

(54) Persistent post-viral cough drug and treatment method

(76) Inventors: Michael H. Eidelman, West Bloomfield, MI (US); Robert E. Weinstein, Farmington Hills, MI (US)

Correspondence Address: Michael H. Eidelman, M.D., 4842 Panorama Circle, West Bloomfield, MI, 48323, US

(21) Appl. No.: 11/190,335

(22) Filed: Jul. 27, 2005

Related U.S. Application Data
(60) Provisional application No. 60/591,792, filed on Jul. 28, 2004.

(51) Int. Cl. A61K 31/573; A61L 9/04

(52) U.S. Cl. 424/45; 514/179

(57) ABSTRACT

A drug for treating persistent cough occurring after an acute viral upper respiratory infection that has a corticosteroid aerosol for inhalation, and labeling to indicate short-term use of the agent for persistent post-viral cough. Also a method for treating persistent post-viral cough that utilizes such a drug.

This application claims the benefit of Provisional Patent Application No. 60/591,792 dated Jul. 28, 2004.

### BACKGROUND OF THE INVENTION

1. Field of the Invention

The present invention relates to medical treatment methods and drugs. Particularly, the present invention relates to the treatment of persistent cough following viral respiratory tract infections.

2. Description of the Prior Art

Acute viral upper respiratory infections (acute viral URI), or “colds” are among the most common ailments. American adults are typically affected once or twice yearly, and children even more frequently. Symptoms include malaise, low-grade fever, rhinorrhea, upper respiratory congestion, sore throat, and cough. Cough that occurs along with the other acute symptoms is considered to be a direct consequence of airway infection.

Symptoms of viral respiratory infections typically last from ten days to two weeks and are almost always self-limited. Acute or short-lived cough, which commonly occurs in association with viral upper respiratory infection, is of little consequence and usually resolves along with other “cold symptoms.” Most individuals self-medicate the symptoms of colds with home remedies or over-the-counter medications such as analgesics and/or decongestants. A wide variety of over-the-counter symptomatic treatments and “cold medicines” are readily available to consumers. Alternatively, some individuals seek professional care, especially when symptoms are severe. The therapeutic armamentarium available to caregivers to treat uncomplicated colds is similarly limited to symptomatic rather than to curative treatments.

The present invention relates to cough that persists well past the usual time of symptom resolution of a viral upper respiratory tract infection. The inventors are physicians who have treated many individuals whose cough has failed to go away as is usually expected, but rather persisted for many weeks after other “cold” symptoms have resolved, and even as long as months afterward. The persistent cough has been observed to be typically dry and hacking, however a moist and/or productive cough has occasionally been observed. The cough is severe in some individuals and may interfere with normal activities or with sleep at night. Affected individuals have typically tried conventional cough medicines such as expectorants, cough suppressants, antihistamines, and decongestants as are found over-the-counter at pharmacies, without satisfactory control of the cough before seeking additional medical care. Many have been prescribed antibiotics at some point during their illness. The persistence and intractability of the cough is often a source of frustration or serious concern to those affected.

Bacterial infections, such as bacterial sinusitis, bronchitis, pneumonia, and otitis media, are known complications of viral upper respiratory infection. Individuals with bacterial infection complicating a viral upper respiratory infection typically experience malaise, general aching, fevers, chills, and/or purulent mucous at the affected site. This is in contrast to the patients herein described with persistent cough, whom otherwise feel generally well and are not subject to the usual symptoms of bacterial infection. It is accepted that conventional anti-bacterial antibiotics are ineffective for viral infection and are best reserved for bacterial infection. Some patients with persistent post-viral cough who suspect superimposed bacterial infection, have sought antibiotic (antibacterial) treatment from us or other medical caregivers. However, in our experience, antibiotic treatments have generally proven unsuccessful in ameliorating the cough. In some instances, cough suppressants, including some of considerable potency such as opiates have been tried, and likewise have not produced satisfactory resolution of cough. The use of opiates for protracted cough is of concern, as these can cause sedation and have the potential for addiction.

The respiratory tract is a continuum from the nose to the pulmonary alveolae. Cough is mediated by the vagus nerves following stimulation of sensory receptors located in and under the epithelium from the larynx to the smaller bronchi. Despite the nomenclature of “viral upper respiratory infection” to signify colds as opposed to viral infection localized predominantly in the lower respiratory tract such as the bronchial tree, or lung parenchyma, it is usual for colds to clinically involve both upper and lower airways. For that reason, a spectrum of nasal, pharyngeal and bronchial symptoms are well known to occur with colds. In the individuals herein described, there is ordinarily nothing unusual about the progression and resolution of cold symptoms along the respiratory tract continuum, except for the occurrence of protracted cough.

Guidelines can be found in the medical literature for the categorization and treatment of cough and as an approach to diagnose and treat cough successfully (Irwin RS and Madison JM. New England Journal of Medicine 2000). Cough of less than three weeks is generally considered “acute” and viral infections of the upper respiratory tract are the most common cause of acute cough. Cough of three to eight weeks duration is categorized as sub-acute, and cough exceeding eight weeks chronic. The persistent post-viral cough herein described therefore fds into the sub-acute or chronic categories. “Postinfectious cough” is defined in this publication as: “cough that begins with an acute respiratory infection that is not complicated by pneumonia (i.e., the chest radiograph is negative) and that ultimately resolves without treatment. It may result from postnasal drip or clearing of the throat due to rhinitis, tracheobronchitis or both, with or without transient bronchial hyperreponsiveness.” Treatment similar to that for common cold is recommended for post-infectious cough if there is a suggestion of post-nasal drip. If such treatment fails, studies to establish sinusitis are recommended. Treatment with decongestants and/or antibiotics is recommended if sinusitis is found. If wheezes or rales are heard, a chest radiograph is advised, and if normal, inhaled bronchodilators and corticosteroids are prescribed. Notably, duration of such treatments is not addressed or advised in this guideline.

The medical literature suggests that post-nasal drip, gastro-esophageal reflux, and asthma, including cough variant asthma, account for the majority of causes of chronic cough. The literature recommends conventional treatments in the event that chronic cough is deemed to be secondary to post-nasal drip, esophageal reflux, or typical asthma. Typical asthma is characterized by wheezing and abnormal pulmonary function tests exhibiting variability of expiratory flow over time and present drug labeling specifies long-term prophylactic use of inhaled corticosteroids when they are used for this entity. Cough variant asthma is a syndrome of cough without the bronchospastic characteristics of typical asthma and is purported to account for some cases of post-infectious cough. The medical literature contains guidelines regarding its treatment. Recommendations include an at-least eight week course of inhaled salbutamol and inhaled beclomethasone (McGarvey, et al, Thorax 1998), a 1 to 2 week trial of oral prednisone followed by the long-term (mean 28 weeks) use of inhaled steroids (Cheryan et al. Annals of Allergy 1994), and the long-term use of inhaled beclomethasome diproprionate (Fujimura et al. Thorax 2003). Nowhere does the medical literature suggest the treatment of either asthma or cough variant asthma, whether following a viral upper respiratory infection or not, with a short-term course of inhaled corticosteroids as in the present invention.

As opposed to what is found in the medical literature, in the usual patient that we have observed and treated for sub-acute or chronic cough after viral respiratory infection, evidence for post-nasal drip, sinusitis, asthma, or gastroesophageal reflux has been lacking. Physical examination of the nose, throat, sinuses, and chest has been unremarkable as have chest x-ray, and simple pulmonary function tests. Antihistamines and decongestants as might be used for post-nasal drip have proven disappointing and have rarely afforded relief of the persistent cough when tried. Antibiotic treatments likewise have been effective to alleviate cough only in rare instances. Rather, we have found that a brief course of inhaled corticosteroids (typically 5 to 7 days) almost always has resulted in remarkably prompt and sustained relief of cough and that recurrence of cough or development of asthmatic symptoms has not occurred. We have been surprised by the regularity with which this treatment has proven effective. Importantly, we have not had to resort to, or subject our patients to prolonged corticosteroid treatments for asthma or cough variant asthma as might be suggested by the literature. In some instances, the resolution of cough with this simple and short treatment has surprised patients who, having suffered a period of persistent and troublesome morbidity, and having tried other treatments without success, have found that “at last something works.” This treatment was discovered despite a medical literature that suggests a variety of causes for protracted cough occurring after viral respiratory infection and suggests other treatments.

Because the respiratory tract is a continuous conduit, topical medications can be applied to the airways surface in aerosol form. The term “aerosol” as used herein is as conventionally defined to indicate either a liquid or solid particle suspended in gas. Many chemical entities in aerosol delivery devices, both liquid and solid, have been developed and marketed for treating respiratory disorders. These include aerosol medications for application to the nose or for inhalation through the mouth. We have utilized a variety of inhaled topical corticosteroid aerosols to treat persistent post-viral cough and have found them all to be generally effective. The following are two examples of aerosols and regimens that we have found effective:
Beclovent Inhalation Aerosol (beclomethasone diprroprionate, USP): two to three sprays t.i.d. for six days.
Pulmicort Turbuhaler® 200 mcg (budesonide powder): one spray t.i.d. for six days.

Drugs for clinical use in the United States are under the scrutiny of the United States Food and Drug Administration (USFDA) and, when approved after evaluation, are labeled to at least convey their indications, dosage, contraindications, side-effects, and other conditions of use. Accordingly, in the United States, a “drug” can be strictly defined as a chemical agent and its associated label. At least at some point between manufacture and dispensing, the chemical agent (or agents) and label are unified and in physical proximity. For example, when drug entities such as tablets, capsules, liquids, aerosols, and other known finished chemical medicament entities are produced for United States consumption, they are packaged with a written label on the packaging container and/or written package insert on or within the container.

All presently marketed inhaled corticosteroids in the United States have labeling that is approved (“indicated”) by the USFDA only for long-term treatment of asthma. For example:

Beclovent (beclomethasone) Inhaler is indicated “only for patients who require chronic treatment with corticosteroids for the symptoms of bronchial asthma. Such patients would include those already receiving systemic corticosteroids, and selected patients who are inadequately controlled on a nonsteroid regimen and in whom steroid therapy has been withheld because of concern over potential adverse effects.

Beclovent Inhalation Aerosol is NOT indicated:
1. For relief of asthma that can be controlled by bronchodilators and other nonsteroid medication
2. In patients who require corticosteroid treatment infrequently.
3. In the treatment of nonasthmatic bronchitis.”

Aerobid® (flunisolide) Inhaler is indicated “in the maintenance treatment of asthma as a prophylactic therapy. Aerobid is also indicated for asthma patients who require systemic corticosteroid administration where adding Aerobid may reduce or eliminate the need for the systemic corticosteroids.”

Azmacort® (triamcinolone acetonide) Inhalation Aerosol is indicated “in the maintenance treatment of asthma as a prophylactic therapy. Azmacort is also indicated for asthma patients who require systemic corticosteroid administration, where adding Azmacort may reduce or eliminate the need for the systemic corticosteroids.”

Flovent® (fluticasone proprionate) Inhalation Aerosol is indicated “for the maintenance treatment of asthma as a prophylactic therapy. It is also indicated for patients requiring oral corticosteroid therapy for asthma. Many of these patients may be able to reduce or eliminate their requirement for oral corticosteroids over time.”

Pulmicort (budesonide) Turbuhaler is indicated for “the maintenance treatment of asthma as prophylactic therapy in adult and pediatric patients six years of age or older. It is indicated for patients requiring oral corticosteroid therapy for asthma. Many of those patients may be able to reduce or eliminate their requirement for oral corticosteroids over time.”

It will be appreciated that in all instances, corticosteroids for inhalation are indicated only for long-term maintenance treatment of asthma in the United States. To our knowledge, no topical anti-inflammatory drug is presently available to caregivers or patients for the acute and limited treatment of persistent post-viral cough as defined in the present invention, even though this condition is not uncommon in our experience. This is particularly striking in view of the recognized inventiveness of the pharmaceutical industry and the strong commercial incentives for new products and for new uses of established ones.

The inventors have improvised treatment for protracted post-viral cough in our patients by utilizing commercially available multi-dose corticosteroid aerosol inhalers that are not indicated for that purpose. These typically provide enough chemical agent for a 29-30 day period. They are neither labeled for short-term use or for treatment of post-viral cough. While we have not encountered medicinal side effects in our treated patients, a general principal of pharmacologic treatment is to avoid overuse of medication. The desirability to minimize treatment and minimize the use of corticosteroids in particular is well recognized. On occasion, the inventors have utilized smaller size inhaler samples with fewer dosing actuations that have been available through the courtesy of pharmaceutical representatives. Although less consistently available, the devices with lesser number of actuations are considered preferable to prevent inadvertent or purposeful overuse of medication. An aerosol device containing a corticosteroid agent for treatment of about two weeks or less is preferred, and about 7 days is most preferred. It is to be noted that, lacking labeling, no inhaled corticosteroid aerosol used experimentally by the inventors comprises a “drug” to treat the persistent post-viral cough patients as herein described.

### SUMMARY OF THE INVENTION

It is an object of the present invention to provide a method to improve the treatment of persistent cough that is known to follow acute viral respiratory tract infections and also to provide a drug to treat that condition. It is a further object of the present invention to teach such methods and drugs as effective alternatives to unnecessary and ineffective treatments that include potentially harmful antimicrobial agents, antitussive agents, systemic corticosteroids, or long term use of topical corticosteroids.

The present invention achieves these and other objectives by teaching a novel method that utilizes a short course of inhaled topical anti-inflammatory medication to treat persistent post-viral cough and by teaching an inhaled corticosteroid drug for the clinical entity herein described.

The following case reports further exemplify the present invention:
1. A 52 year old white male with no previous respiratory history had initial symptoms consisting of nasal congestion, rhinorrhea, post-nasal drip, and dry cough. All symptoms but cough resolved within two week and dry cough persisted for 28 days from onset.

Physical findings: normal nasal and pharyngeal membranes, lack of tenderness in sinus areas, and chest clear to auscultation. Chest x-ray was normal.

PEFR: 104% of predicted. FEV1: 94% of predicted.

Treatment: Beclomethasone MDI 2 sprays (40 mcg@) tid for six days produced significant improvement of cough (slight residual in AM) at one week and subsequent complete resolution. Further medication was not required and the cough did not reoccur.

2. A 48 year old female with no previous respiratory history had initial symptoms consisting of sore throat, nasal congestion, rhinorrhea, post-nasal drip, and productive cough with yellow sputum of nine days duration. She was treated with a five-day course of azithromycin 500 mg per day and all symptoms resolved with the exception of a non-productive “tickling” cough that persisted for 25 days.

Physical findings: normal nasal and pharyngeal membranes, lack of tenderness in sinus areas, and chest clear to auscultation. Chest x-ray was normal.

FEV1: 99% of Normal

Treatment: Beclomethasone MDI 2 sprays (40 mcg@) tid for five days produced complete resolution of cough. Further medication was not required and there was no recurrence of cough.

3. A 61 year old white female with a remote history of self-reported allergic rhinitis with unknown inhalant allergies, but asymptomatic for 10 years and no asthmatic symptoms had the onset of nasal congestion, sore throat, post-nasal drip, and dry cough. Her initial treatment consisted of over-the-counter cold remedies and all symptoms but the cough resolved. A severe dry cough persisted for 22 days.

Physical findings: normal nasal and pharyngeal membranes, lack of tenderness in sinus areas, and chest clear to auscultation. Chest x-ray was normal.

PEFR: 97% of predicted, FEV1: 110% of predicted.

Treatment: Beclomethasone MDI 2 sprays (40 mcg@) tid for five days produced complete resolution of cough and no recurrence.

4. A 58 year old white female with a current history of inhalant allergies to dust, mold and animal dander manifested by rhinitis but no history of asthma or other chest involvement, had initial symptoms consisting of nasal congestion, rhinorrhea, post-nasal drip, malaise, and dry cough. All but the cough resolved within ten days. Severe dry cough persisted for 6 weeks, worse at night. The patient was treated with azithromycin 500 mg per day for five days, without improvement of the cough.

Physical exam revealed normal nasal and pharyngeal membranes and clear chest without wheezing.

Chest x-ray: normal.

PEFR 99% predicted, FEV1 117% predicted.

Treatment with Beclomethasone MDI 2 sprays (40 mcg@) tid for ten days produced complete resolution of cough which did not reoccur.

These above examples characterize what is herein referred to as “persistent post-viral cough syndrome” in that individuals exhibit:
1. initial cold symptoms that subside unremarkably save for persistent cough.
2. lack of remarkable findings upon physical examination of the nose, pharynx, and chest: in particular, an absence of signs for post-nasal drip, sinusitis or bronchospastic disease.
3. lack of remarkable findings on chest x-ray and simple tests of pulmonary function.
4. ready response of persistent cough to a short course of inhaled topical corticosteroids of less than two weeks, 5-7 days usually being sufficient.

In addition to these straightforward and not uncommon events, we have additionally cared for individuals who have a history of smoking and/or respiratory disease, who have also exhibited persistent dry or productive cough triggered by and following acute viral respiratory infection. We have likewise found prompt response to a short course treatment of inhaled corticosteroid in these individuals, for example:

A 69 year old white male cigarette smoker with no history chronic obstructive pulmonary disease symptoms presented with recent nasal congestion, rhinorrhea, sore throat, malaise and productive cough that resolved with OTC cold medication except for a persisting cough productive of white sputum of over three weeks duration. Cough was more severe at night. The patient noted feeling wheezy at times.

Physical exam: revealed normal nasal mucous membranes, absence of sinus tenderness, and the chest was clear to auscultation. Wheezes were not heard. Chest x-ray was normal.

PEFR was 71% of predicted.

Treatment was instituted with Budesonide powder tid for five days. Chest symptoms promptly resolved. No relapse or recurrence of cough has occurred in 8 months of follow up.

### DETAILED DESCRIPTION OF THE PREFERRED EMBODIMENT

The preferred embodiment of the present invention is comprised of an corticosteroid aerosol inhaler device, and a United States Food and Drug Administration approved label that indicates the short-term use of the device for persistent cough following acute viral respiratory infection. As has been noted, many inhalation aerosol devices are presently marketed containing agents as a dry powder or as a particulate liquid. None is labeled to indicate use for treatment of persistent cough following viral upper respiratory tract infections. Examples of preferable labeling might include the following:
“For persistent cough following viral upper respiratory infections or
“For persistent cough following viral upper respiratory infection in the absence of acute bacterial sinusitis.”

Moreover, preferable labels might indicate the use of the corticosteroid aerosol for short-term treatment rather than long-term, as these drugs are presently labeled. Therefore:
“For short-term use for persistent cough following viral upper respiratory infection,” or
“For use for 14 days or less for persistent cough following viral upper respiratory infection,” or
“For use for 5-7 days for persistent cough following viral upper respiratory infection. May be repeated for an additional 5-7 days if needed.”

These examples are not meant to be inclusive and other variations may occur to those skilled in the art and are within the scope of the invention as set forth in the appended claims. Those of skill in the art may also recognize modification to these presently disclosed embodiments. These variations and modifications are meant to be covered by the spirit and scope of the present claims.

What is claimed is:

1. A persistent post-viral cough anti-inflammatory aerosol drug.

2. The drug of claim 1 being a corticosteroid.

3. The drug of claim 1 having a United States Food and Drug Administration approved label stating that the drug is indicated for post-viral cough.

4. The drug of claim 1 wherein the label states that the drug is indicated for short-term treatment.

5. The label of claim 4 wherein said duration is in a range of from five days to two weeks.

6. The quantity of active medicinal agent of claim 1 limited to 14 days of treatment, and preferably to less than 7 days of treatment.

7. A treatment method for persistent post-viral cough comprising the use of a topical anti-inflammatory agent.

8. The treatment method of claim 7, wherein said agent is a corticosteroid.

9. The treatment method of claim 7, wherein said agent is packaged with a United States Food and Drug Administration approved label that states that said agent is indicated for post-viral cough.

10. The treatment method of claim 7, wherein the duration of treatment does not exceed 14 days and is preferably for 7 days or less.

11. A treatment method for persistent post-viral cough comprising the steps of:
a. manufacturing an aerosolized inhalational corticosteroid, and
b. manufacturing a United States Food and Drug Administration approved label that indicates the use of said agent for treatment of persistent post-viral cough.
c. physically unifying said corticosteroid and said label with a packaging container.

12. The treatment method of claim 11 wherein said label contains a recommended duration of treatment in a range of from five days to two weeks.
